(19) United States
(12) Patent Application Publication
(10) Pub. No.: US 2008/0286318 A1
(43) Pub. Date: Nov. 20, 2008

(54) Oral Care Compositions

(75) Inventors: Ralph Spindler, Palatine, IL (US); Stephen J. Urbanec, Arlington Heights, IL (US); Nataliya V. Larionova, Evanston, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMCOL INTERNATIONAL CORPORATION, ARLINGTON HEIGHTS, IL (US)

(21) Appl. No.: 11/815,672

(22) PCT Filed: Feb. 24, 2006
(86) PCT No.: PCT/US2006/006611

§ 371 Date: May 15, 2008

Related U.S. Application Data
(60) Provisional application No. 60/656,276, filed on Feb. 25, 2005.

(51) Int. Cl. A61K 9/14; A61K 8/21; A61K 8/24; A61K 8/22; A61Q 11/00

(52) U.S. Cl. 424/401; 424/49; 424/53; 424/57; 424/52

(57) ABSTRACT

An oral care composition containing polymeric microparticles highly loaded with an oral care compound is disclosed.

### CROSS REFERENCE TO RELATED APPLICATIONS

This application claims the benefit of the filing date of U.S. provisional patent application Ser. No. 60/656,276, filed Feb. 25, 2005.

### FIELD OF THE INVENTION

The present invention relates to an improved delivery system for oral care compounds incorporated into oral care compositions. The delivery system enhances the deposition of and/or improves stability of oral care compounds, such as triclosan, sodium tripolyphosphate, and cetyl pyridinium chloride, in oral care compositions. Other oral care compounds for incorporation into the present oral care compositions include, for example, whitening agents, like sodium percarbonate or sodium perborate; antiplaque deposition aides, like silicone polymers, surfactants, like sodium lauryl sulfate; caries prophylactics, like sodium fluoride, stannous fluoride, and sodium monofluorophosphate; and esthetic agents, like flavors and colors. The oral care composition can be a gel formulation, a paste formulation, or an oral rinse formulation, for example.

### BACKGROUND OF THE INVENTION

Periodontal disease affects a large cross-section of the population, and its impact extends from the loss of teeth to the social embarrassment of mouth odor attributed to an excessive growth of bacteria, especially along the gum line. The solution to this problem often is known, for example, the use of compositions containing antibacterial agents or the incorporation of compounds that help prevent the reattachment of bacteria to the teeth after removal by brushing the teeth. Although such solutions are known, significant challenges still exist with respect to incorporating oral care compounds into an oral care composition such that the stability of the oral care compound is not adversely affected and consumer acceptance of the oral care composition is achieved.

The incorporation of noncationic antimicrobial materials into an oral care composition is disclosed in U.S. Pat. No. 4,894,220. The antimicrobial materials disclosed therein are halogenated diphenyl ethers, like triclosan, and require tuning of the formulation to include solubilizers, such as a high concentration of propylene glycol and/or cosolubilizers, like ethanol, in order to incorporate the water-insoluble triclosan into the composition. Therefore, formulation flexibility is lost by the need to incorporate high concentrations of solubilizing ingredients into the composition.

The use of cyclodextrins as delivery systems for oral care compounds is disclosed in U.S. Pat. No. 5,945,087. Cyclodextrins are known to form inclusion compounds with a variety of small molecules, including halogenated diphenyls, like triclosan. This patent discloses that a combination of menthol, methyl salicylate, thymol, and eucalyptus can be incorporated, with triclosan, into a number of oral care compositions. The effectiveness of this approach is limited because a high concentration of cyclodextrin often is required to effectively solubilize these compounds.

The incorporation of cationic antibacterial agents, like cetyl pyridinium chloride, together with hydrated zinc cations, is disclosed in U.S. Pat. No. 5,948,390. The oral care compositions disclosed therein are reported as stable, although commonly used surfactants in oral care compositions, such as sodium lauryl sulfate, are not incorporated into these compositions.

A method of incorporating a cationic antibacterial agent and surfactants to provide a foaming oral care product is disclosed in U.S. Pat. No. 6,447,758. However, the cationic antibacterial agent and the surfactants are positioned in separate chamber containers, which allow the two components to come in contact with one another during application. Although this arrangement provides an effective product compared to a control formulation, the expense of producing a dual chamber container can be prohibitive, and, therefore, is commercially limiting.

The delivery of oral care compounds through the formation of multicomponent particles, wherein one of the components is a moisture sensitive barrier layer which surrounds nanoparticles composed of wax, active ingredient, and cationic lipids, is disclosed in U.S. Pat. No. 6,589,562.

U.S. Pat. No. 6,696,047 discloses stabilizing sodium chlorite in a variety of oral care compositions, such as toothpastes or oral rinse products. The stabilization of highly reactive sodium chlorite is achieved by ensuring that the pH of the final composition is at least 10 or greater. This is a significant limitation for oral care compositions which may include pH sensitive components, like a polyphosphate.

Delivery systems often are used in personal care and pharmaceutical topical formulations to extend release of an active ingredient, to protect the active ingredient from decomposition in the composition, and/or to enable formulation of the active ingredient into the composition due to difficulties, such as solubility or formulation esthetics. However, a need remains in the art for an efficient delivery system to effectively incorporate oral care compounds into an oral care composition. One type of delivery system that can achieve these attributes in an oral care composition is the adsorbent microparticle delivery systems.

### SUMMARY OF THE INVENTION

The present invention solves a long-standing need for a storage-stable delivery system for oral care compounds in order to provide consumer-acceptable oral care compositions. In particular, the present invention is directed to the use of a microparticle delivery system to extend the delivery of oral care compounds, like functional ingredients and aesthetic agents, from an oral care composition. The present composition also is directed to providing oral care compositions that currently cannot be prepared because of an incompatibility between desired ingredients for inclusion in the composition.

In accordance with the present invention, an oral care compound is loaded onto a microparticle delivery system and the loaded delivery system is incorporated into an oral care composition. The use of a present oral care composition extends the useful life of an oral care compound compared to adding the oral care compound alone to the oral care composition.

Examples of oral care compounds that can be incorporated into the oral care compositions of the present invention include, but are not limited to, antibacterial agents, such as triclosan, cetyl pyridinium chloride, and sodium chlorite; tooth whitening agents, such as hydrogen peroxide, sodium percarbonate, and sodium perborate; antiplaque aides, such as silicone polymers; analgesics, such as benzocaine; and esthetic agents, like flavors and colors, which often are incompatible with other ingredients of the oral care composition. The oral care compositions can be, for example, toothpastes, tooth gels, tooth whiteners, oral analgesics, antiplaque compositions, caries prophylactics, oral antibacterials, oral abrasives, and oral care rinse products.

### DETAILED DESCRIPTION OF THE PREFERRED EMBODIMENTS

As discussed above, it has long been a problem (a) to incorporate a sufficient amount of oral care compound into an oral care composition to provide the desired composition efficacy and esthetics, (b) to stabilize the oral care compound in the oral care composition, (c) to incorporate incompatible oral care compounds into a single oral care compositions, and (d) to provide an extended release of an oral care compound.

The present invention helps overcome these problems by incorporating a high percentage of an oral care compound into a polymeric microparticle delivery system, then including the loaded microparticles in an oral care composition. An oral care compound is incorporated, i.e., loaded, onto the polymeric microparticles by spraying or adding the oral care compound directly to the microparticles in a manner such that an essentially homogeneous distribution of the oral care compound is achieved on the microparticles.

If the oral care compound is a solid, the oral care compound can be dissolved in a suitable volatile solvent. The resulting solution is added to the microparticles, then the volatile solvent is removed, for example, under vacuum with gentle heating. In some cases, this loading process is repeated several times to achieve the desired loading level of the oral care compound on the microparticles. Another method of loading of a solid oral care compound that is insufficiently soluble in an appropriate volatile solvent is to disperse the solid oral care compound in a suitable carrier, such as a polyol, then add the dispersion directly to the microparticle delivery system.

Absorbent polymeric microparticles useful in the present invention have an ability to absorb several times their weight of a liquid compound, such as an oral care compound. One preferred class of adsorbent microparticles is prepared by a suspension polymerization technique, as set forth in U.S. Pat. Nos. 5,677,407; 5,712,358; 5,777,054; 5,830,967; 5,834,577, 5,955,552; and 6,107,429, each incorporated herein by reference (available commercially under the tradename of POLY-PORE E200, INCI name, allylmethacrylate copolymer, from AMCOL International, Arlington Heights, Ill.). Another preferred class of adsorbent microparticles is prepared by a precipitation polymerization technique, as set forth in U.S. Pat. Nos. 5,830,960; 5,837,790, 6,248,849; and 6,387,995, each incorporated herein by reference (sold under the tradename of POLY-POREO L200 by AMCOL International, Arlington Heights, Ill.). These adsorbent microparticles also can be modified after the incorporation of an active compound to modify the rate of release of such a compound, as set forth in U.S. Pat. No. 6,491,953, incorporated herein by reference.

Another useful class of adsorbent polymers prepared by a precipitation polymerization technique is disclosed in U.S. Pat. Nos. 4,962,170; 4,948,818; and 4,962,133, each incorporated herein by reference, and are commercially available under the tradename POLYTRAP from AMCOL International. Other useful, commercially available adsorbent polymers include, for example, MICROSPONGE® (a copolymer of methyl methacrylate and ethylene glycol dimethacrylate), available from Cardinal Health, Sommerset, N.J., and Poly-HIPE polymers (e.g., a copolymer of 2-ethylhexyl acrylate, styrene, and divinylbenzene) available from Biopore Corporation, Mountain View, Calif.

In particular, the adsorbent polymer microparticles prepared by the suspension polymerization technique, e.g., POLY-PORE E200, are a highly porous and highly crosslinked polymer in the form of open (i.e., broken) spheres and sphere sections characterized by a mean unit particle size of about 0.5 to about 3,000 microns, preferably about 0.5 to about 300 microns, more preferably about 0.5 to about 100 microns, and most preferably about 0.5 to about 80 microns. A significant portion of the spheres is about 20 microns in diameter.

The polymeric microparticles are oil and water adsorbent, and have an extremely low bulk density of about 0.008 gm/cc to about 0.1 gm/cc, preferably about 0.009 gm/cc to about 0.07 gm/cc, and more preferably about 0.0095 gm/cc to about 0.04-0.05 gm/cc. The microparticles are capable of holding and releasing oleophilic (i.e., oil soluble or dispersible), as well as hydrophilic (i.e., water soluble or dispersible), active agents, individually, or both oleophilic and hydrophilic compounds simultaneously.

The adsorbent polymer microparticles prepared by the suspension polymerization technique include at least two polyunsaturated monomers, preferably allyl methacrylate and an ethylene glycol dimethacrylate, and, optionally, monounsaturated monomers. The microparticles are characterized by being open to their interior, due either to particle fracture upon removal of a porogen after polymerization or to subsequent milling. The microparticles have a mean unit diameter of less than about 50 microns, preferably less than about 25 microns, and have a total adsorption capacity for organic liquids, e.g., mineral oil, that is at least about 72% by weight, preferably at least about 93% by weight, and an adsorption capacity for hydrophilic compounds and aqueous solutions of about 70% to about 89% by weight, preferably about 75% to about 89% by weight, calculated as weight of material adsorbed divided by total weight of material adsorbed plus dry weight of polymer. In a preferred embodiment, the broken sphere microparticles are characterized by a mean unit diameter of about 1 to about 50 microns, more preferably of about 1 to about 25 microns, most preferably, of about 1 to about 20 microns.

Preferred polymeric microparticle delivery systems comprise a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

Specific polymeric microparticles useful in the present invention can be the previously described POLY-PORE E200, POLY-PORE L200, POLYTRAP, MICROSPONGE, or Poly-HIPE particles, for example. An oral care compound is loaded onto such microparticles to provide microparticles containing about 1% to about 80 wt. %, preferably about 5% to about 70 wt. %, and most preferably about 10% to about 50 wt. %, by weight of the loaded microparticles. The loaded microparticles typically are incorporated into an oral care composition in an amount to provide about 0.05% to about 10%, by weight, of an oral care compound in the composition.

In accordance with the present invention, an oral care compound first is loaded onto the microparticles. Loading of the oral care compound onto the microparticles also is referred to herein as an “entrapment.” The term entrapment refers to a physical loading of the oral care compound onto the polymeric microparticles.

After loading an oral care compound on the microparticles, a barrier layer (i.e., a secondary entrapment), optionally, can be applied to the loaded microparticles to prevent rapid diffusion of oral care compound from the microparticles, and to protect the oral care compound from the surrounding environment until application. This is especially effective for reactive compounds, like cetyl pyridinium chloride, sodium chloride, and sodium tripolyphosphate. Also, the melting point of the barrier layer can be selected such that the barrier layer melts at a higher temperature than the highest temperature that the microparticles will be exposed either during storage or during accelerated aging of the oral care composition.

Examples of materials that can be used as a barrier layer, also termed a secondary loading or secondary entrapment, include, but are not limited to, low melting alcohols (C₈ through C₂₀) and fatty alcohols ethoxylated with one to three moles of ethylene oxide. Examples of fatty alcohols and alkoxylated fatty alcohols include, but are not limited to, behenyl alcohol, caprylic alcohol, cetyl alcohol, cetaryl alcohol, decyl alcohol, lauryl alcohol, isocetyl alcohol, myristyl alcohol, oleyl alcohol, stearyl alcohol, tallow alcohol, steareth-2, ceteth-1, cetearth-3, and laureth-2. Additional fatty alcohols and alkoxylated alcohols are listed in the International Cosmetic Ingredient Dictionary and Handbook, Tenth Edition, Volume 3, pages 2127 and pages 2067-2073 (2004), incorporated herein by reference.

Another class of materials that can be used a barrier layer is the C₈ to C₁₂ fatty acids, including, but not limited to, stearic acid, capric acid, behenic acid, caprylic acid, lauric acid, myristic acid, tallow acid, oleic acid, palmitic acid, isostearic acid and additional fatty acids listed in the International Cosmetic Ingredient Dictionary and Handbook, Tenth Edition, Volume 3, page 2126-2127 (2004), incorporated herein by reference. The barrier material also can be a hydrocarbon, like mineral oil, 1-decene dimer, polydecene, paraffin, petrolatum, vegetable-derived petrolatum or isoparafin. Another class of barrier materials is waxes, like mink wax, carnauba wax, and candelilla wax, for example, and synthetic waxes, like silicone waxes, polyethylene, and polypropylene, for example.

Fats and oils can be useful barrier material agents, which include, for example, but are not limited to, lanolin oil, linseed oil, coconut oil, olive oil, menhaden oil, castor oil, soybean oil, tall oil, rapeseed oil, palm oil, and neatsfoot oil, and additional fats and oils listed in the International Cosmetic Ingredient Dictionary and Hand-book, Tenth Edition, Volume 3 (2004), pages 2124-2126. Other useful classes of barrier materials include a water-insoluble ester having at least 10 carbon atoms, and preferable 10 to about 32 carbon atoms. Numerous esters are listed in International Cosmetic Ingredient Dictionary and Handbook, Tenth Edition, pages 2115-2123 (2004).

Alternatively, an oral care compound can be mixed with a barrier layer material, then loaded on a microparticle delivery system. In the case of liquid oral care compounds, the materials disclosed above as barrier materials also can be used as an additive for thickening the liquid oral care compound, and thereby minimize premature diffusion of the oral care compound from the polymeric microparticle.

The barrier layer can be about 10% to about 70%, by total weight of the loaded polymeric microparticles. In a preferred embodiment, the barrier layer is present at about 25% to about 50 wt. %, by total weight of the loaded polymeric microparticles.

An oral care composition of the present invention therefore comprises polymeric microparticles loaded with an oral care compound and an optional barrier material. The oral care composition also can contain other ingredients well known in the oral care arts.

An oral care compound is loaded into the polymeric microparticles in an amount to provide microparticles containing about 1% to about 80%, preferably about 5% to about 70%, and more preferably about 10% to about 50%, of the oral care compound, by weight of the loaded microparticles. In one embodiment, the oral care compound is loaded onto the polymeric microparticles in an amount of up to about 80%, by weight of the loaded microparticles. For example, a flavor can be incorporated in an amount of about 1% to about 80% by weight of the loaded microparticles.

As used herein, the term “loaded microparticle” refers to a microparticle having an ingredient added thereto. Loading of the ingredient includes one or more of impregnating, imbedding, entrapping, absorbing, and adsorbing of the ingredient into or onto the polymeric microparticles.

A variety of oral care compounds can be incorporated into the polymeric microparticles. The oral care compounds include, but are not limited to:

(a) antibacterials, such as a halogenated diphenyl ethers, e.g., 2′,4,4′-trichloro-2-hydroxydiphenyl ether, known under the trade name triclosan, and 2,2′-dihydroxy-5,5′-dibromo-diphenyl ether; 2,2′-methylenebis-4-4-chloro-6-bromo-phenol); halogenated salicylanilides; halogenated carbanilides; sodium tripolyphosphate; cetyl pyridinium chloride; benzalkonium chloride; sodium hypochlorite; hexachlorophene; thymol; cresols; guaiacol; eugenol; creosote; copper sulphate; copper-(ethyl) maltol; zinc- and stannous salts, such as zinc citrate and sodium zinc citrate; stannous pyrophosphate; and sanguinarine extract;

(b) caries prophylactics, such as a fluoride ion source like sodium fluoride, stannous fluoride, and sodium monofluorophosphate; sodium chloride; and sodium bicarbonate;

(c) a tooth whitener, such as hydrogen peroxide, sodium percarbonate, sodium perborate, potassium peroxydiphosphate, and organic peracids;

(d) an antiplaque agent, such as a silicone polymer;

(e) an analgesic, such as codeine, aspirin, acetaminophen, propoxyphene, meperidine, and benzocaine;

(f) flavors, such as spearmint oil, methyl salicylate, cinnamon oil, peppermint oil, clove oil, saccharin, thymol, menthol, and eucalyptus; and

(g) surfactants, such as sodium lauryl sulfate.

The loaded microparticles are included in an oral care composition. As stated above, the oral care composition comprises about 0.05% to about 50%, and often about 0.1% to about 25%, by weight, of the loaded microparticles. The oral care composition can be, for example, a tooth paste, an oral rinse, an antibacterial, a caries prophylactic, a tooth whitener, an antiplaque composition, an abrasive, or an analgesic.

The loaded microparticles are included in an oral care composition. As stated above, the oral care composition comprises additional ingredients well know in the art and selected with the final end use of the composition in mind. The loaded microparticles are included in the oral care composition in a sufficient amount to provide about 0.05% to about 10%, and preferably about 0.1% to about 5% of the oral care compound, by weight of the oral care composition.

The oral care composition typically contains optional ingredients to perform a desired function or provide an esthetic effect. The optional ingredients are included in an oral care composition in a sufficient amount to perform their intended function. Nonlimiting examples of optional ingredients commonly used in oral care compositions are polyols, e.g., glycerin and propylene glycol, a gum, e.g., tragacanth, karaya gum, and carboxymethylcellulose, a filler, e.g., pumice, kaolin, an opacifying agent, a buffering agent, a dye, a preservative, a carrier, e.g., starch or sucrose, a particulate abrasive material, e.g., silica, alumina, calcium carbonate, dicalcium phosphate, calcium pyrophosphate, hydroxyapatite, trimetaphosphate, and insoluble hexametaphosphate, thickeners, e.g., synthetic polymers such as polyacrylates and carboxyvinyl polymers, vitamins, e.g., Vitamin C and plant extracts, desensitizing agents, e.g., glycerol mono oleate, potassium citrate, potassium chloride, potassium tartrate, potassium bicarbonate, potassium oxlate and potassium nitrate, and plaque buffers, e.g., urea, calcium lactate, calcium glycerophosphate, and strontium polyacrylate.

### EXAMPLES

### Example 1

Loading of Triclosan. To 37.5 g of isopropyl alcohol was added 12.5 g of triclosan (IRGACARE MP, Ciba). The solution was stirred until the triclosan was completely solubilized. The loading solution was added slowly to 50 g of POLYTRAP with sufficient stirring and for an extended period of time to ensure that the loading was homogeneous. The loaded POLYTRAP was placed in a vacuum oven at 45° C. and dried until the isopropyl alcohol was essentially completely removed. This loading process was repeated three additional times until the final load of triclosan in the POLYTRAP was equal to weight of the polymer resulting in a 1:1 load of triclosan in POLYTRAP.

### Example 2

To 25 g of the 1:1 loaded triclosan described in Example 1 was added 37.5 g of shea butter that first was melted at 80° C., then cooled to 45° C., before addition to the loaded POLY-TRAP in a stepwise process which provided a final composition containing 20% triclosan, 20% POLYTRAP and 60% shea butter, by weight.

### Example 3

To 15 g of the triclosan loading described in Example 1 was added 30 g of a solution containing 1:1 blend of dimethicone (60,000 cst) and hexanes. The solution was added in step-wise process with sufficient agitation to provide a homogeneous loading. The resulting loaded microparticles then were placed in a vacuum oven at 40° C. overnight to give a final composition containing 25% POLYTRAP, 25% triclosan, and 50% dimethicone, by weight.

### Example 4

A solution containing 10 g sodium tripolyphosphate was added to 100 g of deionized (DI) water, then the resulting solution was stirred until homogeneous. The solution was added to 100 g of POLY-PORE® E200 microparticles in a stepwise process with sufficient stirring to ensure that the loading solution was homogeneously distributed. The resulting product was placed in a vacuum oven at 50° C., then the material was dried until essentially all the water was removed. A second loading solution was prepared containing the same ratios of sodium tripolyphosphate and water as above, and this solution was added to the dried loaded POLY-PORE® E200 particles in a similar step-wise process. The resulting loaded microparticles were placed in the vacuum oven at 50° C., then dried until the water was essentially completely removed. The final composition contained 16.7% sodium tripolyphosphate and 83.3% POLY-PORE® E200, by weight.

### Example 5

A dispersion of sodium percarbonate in polyethylene glycol (PEG, MW ca. 400) was prepared by adding 125.25 g of sodium percarbonate to 254.29 g of PEG. The components were mixed with a dispersion blade at sufficient speed to ensure that the sodium percarbonate was uniformly admixed with the PEG. To 91.4 g of POLYTRAP was added 365.5 g of the sodium percarbonate dispersion. The dispersion was slowly added in a stepwise process with sufficient mixing to ensure that loading was homogeneous. The final composition contained 26% sodium percarbonate, 54% PEG, and 20% POLYTRAP, by weight.

### Example 6

A cetyl pyridinium chloride loading was prepared by first dissolving 60 g of cetyl pyridinium chloride in 240 g of denatured ethanol, then stirring the mixture until the cetyl pyridinium chloride was completely dissolved. The resulting solution then was added to 100 g of POLY-POREO E200 in a stepwise fashion with sufficient mixing to ensure that the loading solution was completed dispersed onto the polymer. The resulting loaded delivery system was placed in a vacuum oven and dried at 50° C. under vacuum until essentially all the solvent was removed. The final composition contained 37.5% cetyl pyridinium chloride and 62.5% POLY-POREO E200, by weight. Similar loaded microparticles were prepared by substituting POLY-POREO E200 with POLYTRAP.

### Example 7

To 10 g of a loading of 37.5% cetyl pyridinium chloride on POLYTRAP was added 10 g of stearyl alcohol that first was heated to 80° C. The stearyl alcohol was added to the loaded POLYTRAP in a stepwise process using sufficient stirring to ensure that the microparticles were uniformly coated. The final composition contained 18.7% cetyl pyridinium chloride, 50% stearyl alcohol, and 31.3% POLYTRAP, by weight. A similar loading was prepared wherein the final composition contained 12.4% cetyl pyridinium chloride, 67% stearyl alcohol, and 20.6% POLYTRAP, by weight.

### Example 8

To 72 g of a 37.5% loading of cetyl pyridinium chloride on POLYTRAP was added 144 g of shea butter that first was melted at 80° C., then added in a stepwise process with sufficient stirring to homogeneously incorporate the shea butter throughout the loaded POLYTRAP. The final composition contained 12.4% cetyl pyridinium chloride, 67% shea butter, and 20.6% POLYTRAP, by weight.

### Example 9

A loading of dimethicone (50 cst) in POLYTRAP was prepared by directly adding 400 g of dimethicone to 100 g of POLYTRAP to provide a composition that contained 80% dimethicone and 20% POLYTRAP, by weight.

### Example 10

A loading of peppermint flavor on POLY-POREO E200 was prepared by adding 140.5 g of peppermint flavor (Bell Flavors & Fragrances) to 46.8 g of POLY-PORE®. The oil was added in a step-wise process with sufficient mixing to ensure that a homogeneous loading of the oil on the microparticles.

### Example 11

Toothpaste base

| | | |
|---|---|---|
| Phase A | Betaine | 2.0 wt. % |
| | Sorbitol | 24.5 wt. % |
| | Sodium citrate | 0.2 wt. % |
| | Polyethylene glycol | 2.0 wt. % |
| | (MW 1500) | |
| | DI (deionized) Water | 49.1 wt. % |
| Phase B | Cellulose gum | 0.5 wt. % |
| Phase C | Sorbitol | 1.5 wt. % |
| | DI Water | 0.5 wt. % |
| | Pigment White 6 | 1.0 wt. % |
| Phase D | Zeodent 113 (Huber) | 10.0 wt. % |
| | Zeodent 116 (Huber) | 7.0 wt. % |
| Phase E | Sodium lauryl sulfate | 1.7 wt. % |

Heat Phase A to 45° C., then add Phase B slowly with stirring. Mix Phase C components together, and add to the mixture of Phases A and B. Add Zeodent materials of Phase D, then add Phase E with stirring.

### Example 12

To 50 g of the toothpaste base of Example 11 was added 1.2 g of the triclosan loaded microparticles of Example 3 with stirring.

Obviously, many modification and variations of the invention as hereinbefore set forth can be made without department from the spirit and scope thereof and, therefore, only such limitations should be imposed as are indicated by the appended claims.

What is claimed is:

1. An oral care composition comprising an oral care compound loaded onto polymeric microparticles.

2. The oral care composition of claim 1 wherein the oral care compound comprises an antibacterial agent, a flavor, a tooth whitener, a caries prophylactic, an antiplaque agent, a surfactant, an analgesic, or a mixture thereof.

3. The oral care composition of claim 1 wherein the polymeric microparticles are highly crosslinked and are derived from methacrylate monomers, acrylate monomers, or mixtures thereof.

4. The oral care composition of claim 1 wherein the polymeric microparticles comprise an allyl methacrylate copolymer, an ethylene glycol dimethacrylate/allyl methacrylate copolymer, a lauryl methacrylate/ethylene glycol dimethacrylate copolymer, and mixtures thereof.

5. The oral care composition of claim 1 wherein the polymeric microparticles are selected from the group consisting of a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

6. The oral care composition of claim 1 wherein the polymeric microparticles comprise a copolymer of allyl methacrylate and ethylene glycol dimethacrylate, a copolymer of ethylene glycol dimethacrylate and lauryl methacrylate, or a mixture thereof.

7. The oral care composition of claim 6 wherein the polymeric microparticles comprise copolymer of ethylene glycol dimethacrylate and lauryl methacrylate.

8. The oral care composition of claim 2 wherein the antibacterial agent comprises triclosan, benzalkonium chloride, or cetyl pyridinium chloride.

9. The oral care composition of claim 2 wherein the whitening agent comprises hydrogen peroxide, sodium percarbonate, sodium perborate, potassium peroxydiphosphate, an organic peracid, or mixtures thereof.

10. The oral care composition of claim 1 wherein the oral care compound is present in an amount of about 1% to about 80%, by weight of the loaded microparticles.

11. The oral care composition of claim 10 wherein the oral care compound is present in an amount of about 5% to about 70%, by weight of the loaded microparticles.

12. The oral care composition of claim 11 wherein the oral care compound is present in an amount of about 10% to about 50%, by weight of the loaded microparticles.

13. The oral care composition of claim 2 comprising a flavor in an amount of about 1% to about 80%, by weight of the loaded microparticles.

14. The oral care composition of claim 2 wherein the antibacterial agent, the tooth whitener, or the caries prophylactic is present in an amount of about 5% to about 70%, by weight of the loaded microparticles.

15. The oral care composition of claim 14 wherein the antibacterial agent, the tooth whitener, or the caries prophylactic is present in an amount of about 10% to about 50%, by weight of the loaded microparticles.

16. The oral care composition of claim 1 wherein the loaded microparticles further comprise a barrier layer.

17. The oral care composition of claim 16 wherein the barrier layer is present in an amount of about 10% to about 70%, by total weight of the loaded microparticles.

18. The oral care composition of claim 17 wherein the barrier layer is present in an amount of about 20% to about 50%, by total weight of the loaded microparticles.

19. The oral care composition of claim 1 wherein the loaded microparticles are present in the composition in an amount of about 20% to 80%, by weight, of the oral care composition.

20. The oral care composition of claim 1 wherein the oral care compound is present in the composition in an amount of about 0.05% to about 50%, by weight, of the oral care composition.

21. The oral care composition of claim 20 wherein the oral care compound is present in the composition in an amount of about 0.1% to about 25%, by weight, of the oral care composition.

22. The oral care composition of claim 1 wherein the composition is a toothpaste, an oral rinse, a tooth whitener, an oral analgesic, an oral antibacterial, a caries prophylactic, an abrasive, or an anti-plaque composition.

23. The oral care composition of claim 1 wherein the oral care compound is selected from the group consisting of triclosan, sodium tripolyphosphate, sodium chlorite, cetyl pyridinium chloride, hexachlorophene, eugenol, benzalkonium chloride, hydrogen peroxide, sodium percarbonate, sodium perborate, sodium lauryl sulfate, sodium fluoride, stannous fluoride, sodium monofluorophosphate, a silicone polymer, a flavor, a color, benzocaine, meperidine, and mixtures thereof.
